(19) United States
(12) Patent Application Publication
(10) Pub. No.: US 2007/0259415 A1
(43) Pub. Date: Nov. 8, 2007

(54) Polyacryamide beads containing encapsulated cells

(75) Inventors: Jana Zigova, Visp (CH); Karen Robins, Visp (CH); Johannes Bartek, Visp (CH)

Correspondence Address: NIXON & VANDERHYE, PC, 901 NORTH GLEBE ROAD, 11TH FLOOR, ARLINGTON, VA, 22203, US

(73) Assignee: LONZA Ltd., Basel (CH)

(21) Appl. No.: 11/822,928

(22) Filed: Jul. 11, 2007

Related U.S. Application Data
(63) Continuation of application No. 10/872,778, filed on Jun. 22, 2004, now abandoned.

(30) Foreign Application Priority Data
Oct. 27, 2003 (EP) 03024648.2

(51) Int. Cl. C12N 1/21

(52) U.S. Cl. 435/243

(57) ABSTRACT

Polyacrylamide beads containing encapsulated cells were prepared by a process comprising the steps of (i) providing an aqueous solution of a mixture of acrylic monomers, (ii) providing a suspension of cells in an aqueous solution of a persulfate (iii) providing an emulsion of an aqueous solution of a tertiary amine in a water-immiscible liquid, which liquid optionally contains a surfactant, (iv) mixing the solution provided in step (i) and the suspension provided in step (ii) (v) adding the mixture obtained in step (iv) to the stirred emulsion provided in step (iii) (vi) polymerizing the mixture of acrylic monomers and simultaneously encapsulating the cells to form polyacrylamide beads containing encapsulated cells.

The present application is a continuation of U.S. application Ser. No. 10/872,778, filed Jun. 22, 2004 (pending), which claims benefit of EP Application No. 03024648.2, filed 27 Oct. 2003, the entire contents of each of which is hereby incorporated by reference in this application.

### DESCRIPTION

The present invention refers to polyacrylamide beads containing encapsulated cells, to a process for their preparation and to their use as a biocatalyst.

Polyacrylamide beads containing encapsulated cells can be used as a biocatalyst for various biotransformations depending on the enzymes contained within the cells. For example polyacrylamide beads containing encapsulated bacterial cells of a strain of the genus Rhodococcus containing a nitrile hydratase can be used for the transformation of nitriles to amides.

Polyacrylamide beads containing enzymes have been described by Nilsson et al. (Biochim. Biophys. Acta 1972, 268, 253-256). A solution of ammonium persulfate (0.25 g, 1.1 mmol) in triethanolamin-HCl buffer (0.05 M, pH 7.0, 0.5 mL) and N,N,N′,N′-tetramethyl-ethylenediamine (0.5 mL, 0.385 mg, 3.3 mmol) were added to a solution (60 mL) of trypsin (60 mg), acrylamide (8.55 g, 120 mmol) and N,N′-methylenebisacrylamide (0.45 g, 2.9 mmol) in triethanolamin-HCl buffer (0.05 M, pH 7.0). The solution was poured into a stirred organic phase (toluene/chloroform 290:110, 400 mL) containing sorbitan sesquioleate (1 mL). The polymerization was carried out at 4° C. for 30 min. Nilsson et al. (Biochim. Biophys. Acta 1972, 268, 253-256) does not describe the encapsulation of cells in polyacrylamide beads.

Mosbach et al. (U.S. Pat. No. 4,647,536 A) describes the preparation of various bead polymers containing encapsulated cells wherein an animal oil, a vegetable oil, tri-butylphosphate, liquid silicone, paraffin oil or phthalic acid dibutyl ester was used as the water-insoluble phase. Polyacrylamide beads containing yeast cells or enzymes were prepared by dissolving acrylamide (17.6 g, 248 mmol) and N,N′-methylenebisacrylamide (1.2 g, 8 mmol) in tris-buffer (100 mL, 0.05 M, pH 7), mixing 8 mL of this solution with yeast cells or enzymes (e.g. peroxidase, 10 mg/mL, 2 mL) and ammonium persulfate (0.4 g/mL, 20 μL (8 mg, 0.03 mmol)) and dispersing the mixture in soybean oil (40 mL). N,N,N′,N′-Tetra-methylethylenediamine (100 μL, 77.0 mg, 0.66 mmol) was added when a suitable bead size had been reached.

It is an object of the present invention to provide polyacrylamide beads containing cells and a process for their preparation.

This object is achieved by the polyacrylamide beads of claim 12 and by the process of claim 1.

The process of the present invention for the preparation of polyacrylamide beads containing encapsulated cells comprises the steps of
(i) providing an aqueous solution of a mixture of acrylic monomers,
(ii) providing a suspension of cells in an aqueous solution of a persulfate
(iii) providing an emulsion of an aqueous solution of a tertiary amine in a water-immiscible liquid, which liquid optionally contains a surfactant,
(iv) mixing the solution provided in step (i) and the suspension provided in step (ii)
(v) adding the mixture obtained in step (iv) to the stirred emulsion provided in step (iii), and
(vi) polymerizing the mixture of acrylic monomers and simultaneously encapsulating the cells to form polyacrylamide beads containing encapsulated cells.

The process of the present invention is advantageous insofar as the tertiary amine is already added to the water-immiscible liquid before the addition of the acrylic monomers, the cells and the persulfate.

The polyacrylamide beads formed by the process of the present invention are of spherical or almost spherical shape.

The polyacrylamide beads can have a size of 0.01 to 5 mm and a mechanical strength of at least 10 mN. Preferably, the polyacrylamide beads have a size of 0.05 to 3 mm and a mechanical strength of at least 200 mN. More preferably the polyacrylamide beads have a size of 0.1 to 1.5 mm and a mechanical strength of at least 300 mN.

The mechanical strength is measured by applying pressure to a bead which is placed between two plates until the bead breaks.

The cell can be a bacterial cell, a fungal cell, a yeast cell, a plant cell or a mammalian cell. Preferably, the cell is a bacterial cell more preferably it is a cell of a bacterium of the group nocardioform Actinomycetes or of a bacterium of the family Enterobacteriaceae. Even more preferably the cell is a cell of a bacterium of the genera Rhodococcus or Escherichia, and most preferably it is a cell of a bacterium of the genus Rhodococcus.

Examples of bacteria are gram-positive bacteria such as bacteria of the genera Bacillus, Acetobacterium, Actinomyces, Arthrobacter, Corynebacterium, Gordona, Nocardia, Rhodococcus or Amycolatopsis, and gram-negative bacteria such as bacteria of the genera Acetobacter, Agrobacterium, Alcaligenes, Comamonas, Gluconobacter, Pseudomonas, Rhizobium, Citrobacter, Enterobacter, Escherichia or Klebsiella.

Examples of bacteria of the group nocardioform Actinomycetes are bacteria of the genera Gordona, Nocardia, Rhodococcus and Amycolatopsis. Examples of bacteria of the family Enterobacteriaceae are bacteria of the genera Citrobacter, Enterobacter, Escherichia and Klebsiella.

The cells can be cultivated by methods known in the art.

The bacterial cell can contain the gene encoding the enzyme of interest on the chromosome or can be transformed with a plasmid containing the gene encoding the enzyme of interest.

If the bacterial cells contains the gene encoding the enzyme of interest on the chromosome, and this enzyme is a catabolic enzyme, the bacterial cell can be cultivated in the presence of a suitable enzyme inducer. For example, cells of a strain of the genus Rhodococcus can be cultivated in the presence of a nitrile hydratase inducer to induce the expression of a nitrile hydratase. Examples of suitable inducers for a nitrile hydratase of a stain of the genus Rhodococcus are methacrylamide, crotonamide and propionamide.

If the bacterial cells are transformed with a plasmid containing the gene encoding the enzyme of interest, and this gene is under the control of an inducible promoter, the transcription of the gene encoding the enzyme of interest can be induced at a suitable point of time during the cultivation. Examples of inducible promoters are the trp, the lac, the tac, the arabinose and the rhamnose promoter. The induction depends on the promoter employed. For example, the rhamnose promoter can be induced by addition of L-rhamnose.

After cultivation, the cells containing the enzyme of interest can be separated from the fermentation broth. Preferably the cells stored in an appropriate buffer below 5° C.

The mixture of acrylic monomers can consist of at least one monofunctional and at least one bifunctional acrylic monomer.

A monofunctional acrylic monomer can be a monomer of the formula 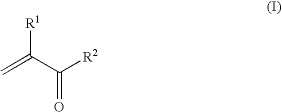
wherein
R¹ is H or methyl,
R² is selected from the group consisting of NH₂, NHR³, N(R³)₂, NH—(CH₂)ₙ—N(R³)₂ and O—(CH₂)ₙ—N(R³)₂
R³ at each occurrence is C₁₋₄-alkyl, and
n is an integer from 1 to 4.

Examples of monofunctional acrylic monomers are acrylamide (R¹═H, R²═NH₂), methacrylamide (R¹=methyl, R²═NH₂), N-alkylacrylamides R¹═H, R²═NHR³, R³═C₁₋₄-alkyl) such as N-ethylacrylamide (R³=ethyl), N-isopropylacrylamide (R³=isopropyl) or N-tert-butylacrylamide (R³=tert-butyl), N-alkylmethacrylamides (R¹=methyl, R²═NHR³, R³═C₁₋₄-alkyl) such as N-ethylmethacrylamide (R³=ethyl) or N-isopropylmethacrylamide (R³=isopropyl), N,N-dialkylacrylamides (R¹═H, R²═N(R³)₂, R³═C₁₋₄-alkyl) such as N,N-dimethylacrylamide (R³=methyl) and N,N-diethyl-acrylamide (R³=ethyl), N-[(dialkylamino)alkyl]acrylamides (R¹═H, R²═NH—(CH₂)ₙ—NH(R³)₂, R³═C₁₋₄-alkyl) such as N-[3-dimethylamino)propyl]acrylamide (n=3, R³=methyl) or N-[3-(diethylamino)propyl]acrylamide (n=3, R³=ethyl), N-[(dialkylamino)alkyl]methacrylamides (R¹=methyl, R²═NH—(CH₂)ₙ—NH(R³)₂, R³═C₁₋₄-alkyl) such as N-[3-(dimethylamino)propyl]methacrylamide (R³=methyl) or N-[3-(diethylamino)propyl]methacrylamide (R³=ethyl), (dialkylamino)alkyl acrylates (R¹═H, R²═O—(CH₂)ₙ—NH(R³)₂, R³═C₁₋₄-alkyl) such as 2-(dimethylamino)ethyl acrylate (n=2, R³=methyl), 2-(dimethylamino)propyl acrylate (n=3, R³=methyl) or 2-(diethylamino)ethyl acrylates (n=2, R³=ethyl) and (dialkylamino)alkyl methacrylates (R¹=methyl, R²═O—(CH₂)ₙ—NH(R³)₂, R³═C₁₋₄-alkyl) such as 2-(dimethylamino)ethyl methacrylate (n=2, R³=methyl).

N-Allylacrylamides, N-alkylmethacryamides, N,N-dialkylacrylamides, N,N-dialkyl-methacrylamides, N-[(dialkylamino)alkyl]acrylamides, N-[(dialkylamino)-alkyl]methacrylamides, (dialkylamino)alkyl acrylates and (dialkylamino)alkyl acrylates can be prepared by methods known in the art, for example by reacting acryloyl chloride, methyl acrylate, methacryloyl chloride or methyl methacrylate with the respective alkylamine, dialkylamine or (dialkylamino)alkylamine or (dialkylamino)alcohol.

Bifunctional acrylic monomers can be monomers of the formula 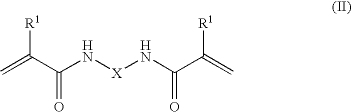
wherein

R¹ is H or methyl

—X— is —(CH₂)ₙ— or —(CH—OH)ₙ—

n is an integer from 1 to 4

Examples of bifunctional acrylic monomers are N,N′-methylenebisacrylamide (R¹═H, —X—═—(CH₂)ₙ—, n=1), N,N′-methylenebismethacrylamide (R¹=methyl, —X—═(CH₂)ₙ, n=1), N,N′-ethylenebisacrylamide (R¹═H, —X—═—(CH₂)_{n—, n=}2), N,N′-ethylenebis-methacrylamide (R=methyl, —X—═—(CH₂)ₙ—, n=2), N,N′-propylenebisacrylamide (R¹═H, —X—═—(CH₂)ₙ—, n=3) and N,N′-(1,2-dihydroxyethylene)bisacrylamide (R¹═H, —X—═—(CH—OH)ₙ—, n=2)

Bifunctional acrylic monomers can be prepared by methods known in the art, for example bifunctional acrylic monomers where —X— is —CH₂)ₙ—, can be prepared by reacting acryloyl chloride, methyl acrylate, methacryloyl chloride or methyl methacrylate with the respective diamine.

Preferably, the bifunctional acrylic monomer is selected from the group consisting of N,N′-methylenebisacrylamide, N,N′-methylenebismethacrylamide and N,N′-(1,2-di-hydroxyethylene)bisacrylamide, and the monofunctional monomer is selected from the group consisting of acrylamide, methacrylamide, N,N-dialkylacrylamides, N-[(dialkyl-amino)alkyl]methacrylamides, (dialkylamino)alkyl acrylates and (dialkylamino)alkyl methacrylates.

More preferably, the bifunctional acrylic monomer is N,N′-methylenebisacrylamide, and the monofunctional monomer is selected from the group consisting of acrylamide, N,N-dimethylacrylamide, N-[3-dimethylamino)propyl]methacrylamide and 2-(dimethylamino)ethyl methacrylate.

The persulfate can be any water-soluble persulfate. Examples of water soluble persulfates are ammonium persulfate and alkali metal persulfates. Examples of alkali metals are lithium, sodium and potassium. Preferably, the persulfate is ammonium persulfate or potassium persulfate, more preferably, it is ammonium persulfate.

The tertiary amine can be any water-soluble tertiary amine. Preferably, the tertiary amine is N,N,N′,N′-tetramethylethylenediamine or 3-dimethylamino)propionitrile, more preferably it is N,N,N′,N′-tetramethylethylenediamine.

The water-immiscible liquid can be any water-immiscible material that is liquid at the temperature of polymerization. Examples of water-immiscible liquids are mineral oils, vegetable oils and synthetic oils. Examples of mineral oils are toluene, xylene, dearomatized hydrocarbon mixtures such as Exxsol D100 and isoparaffine mixtures such as Isopar M. Examples of vegetable oils are sunflower oil, olive oil, peanut oil, almond (oil, safflower oil, soybean oil and corn oil. An example of a synthetic oil is silicone oil.

Preferably the water-immiscible liquid is a mineral oil. More preferably, it is a saturated hydrocarbon or a mixture thereof. Most preferably it is a dearomatized hydrocarbon mixture or an isoparaffin mixture.

The water-immiscible liquid can optionally contain a surfactant. The surfactant can be any suitable surfactant Examples of suitable surfactants are nonionic surfactants such as sorbitan fatty acid esters, polyethyleneglycol fatty acid esters, ethyleneglycol fatty acid esters or glycerol fatty acid esters and cationic surfactants such as tetraalkyl ammonium salts, wherein at least one of the alkyls has at least 8 carbon atoms. Examples of fatty acids are oleic acid or stearic acid Examples of alkyl are ethyl, propyl and butyl. Examples of alkyls having at least 8 carbons are octyl, nonyl and decyl.

The ratio of surfactant/oil can be up to 0.10:1 (w/w). Preferably, no surfactant is used.

An aqueous solution of a mixture of acrylic monomers can be provided by dissolving the acrylic monomers in water or a buffer. A suspension of cells in an aqueous solution of a persulfate can be provided by mixing a solution of a persulfate in water or a buffer with a suspension of the cells in water or a buffer. Preferably the acrylic monomers are dissolved in and the cells are suspended in a buffer, and the pH is adjusted to a pH within the range from 5 to 10 which is favored by the enzyme of interest For example a pH within the range from 6 to 8 is favored by a nitrile hydratase from a strain of the genus Rhodococcus.

An emulsion of an aqueous solution of a tertiary amine in a water-immiscible liquid can be provided by emulsifying a solution of a tertiary amine in water or a buffer in the water-immiscible liquid.

Preferably, the aqueous solution of a mixture of acrylic monomers, the suspension of cells in an aqueous solution of a persulfate and the emulsion of an aqueous solution of a tertiary amine in the water-immiscible liquid, which liquid optionally contains a surfactant, are deoxygenated, e.g. by purging with nitrogen.

The aqueous solution of a mixture of acrylic monomers and the suspension of cells in an aqueous solution of a persulfate are mixed and immediately dropped into the stirred emulsion of an aqueous solution of a tertiary amine in the water-immiscible liquid. Examples of suitable stirrers are three or four pitch bladed turbine stirrers, propeller stirrers or visco-jet® stirrers. Preferably, a visco-jet® stirrer is used. Preferably, the polymerization is carried out at 5 to 35° C. More preferably it is carried out at 15 to 25° C., and most preferably it is carried out at 18 to 22° C.

Following ratios are preferably applied for the polymerization step:

Preferably ratio of the mixture of acrylic monomers/water is 0.05:1 to 0.5:1 (w/w). More preferably it is 0.1:1 to 0.3:1 (w/w). Most preferably it is 0.2:1 to 0.28:1 (w/w).

Preferably the ratio of bifunctional acrylic monomers/monofunctional acrylic monomers is 0.001:1 to 0.8:1 (mol/mol). More preferably it is 0.01:1 to 0.08:1 (mol/mol). Most preferably it is 0.03:1 to 0.06:1 (mol/mol).

Preferably ratio of dry cells/mixture of acrylic monomers is 0.001:1 to 1:1 (w/w). More preferably it is 0.2:1 to 0.9:1. Even more preferably it is 0.4 to 0.8:1 (w/w). Most preferably it is 0.5:1 to 0.7:1 (w/w).

Preferably the ratio of persulfate/mixture of acrylic monomers is 0.0001:1 to 0.1:1 (mol/mol). More preferably it is 0.001:1 to 0.05:1 (mol/mol). Most preferably it is 0.002:1 to 0.03:1 (mol/mol).

Preferably ratio of tertiary amine/persulfate is 0.2:1 to 50:1 (mol/mol). Preferably it is 0.8:1 to 10:1 (mol/mol). Most preferably it is 1:1 to 5:1 (mol/mol).

Preferably ratio of oil/water is 1.2:1 to 10:1 (w/w). More preferably it is 1.3:1 to 7:1 (w/w). Even more preferably it is 1.4:1 to 5:1 (w/w). Most preferably it is 1.5:1 to 4:1 (w/w).

Preferably, the polyacrylamide beads obtained after the polymerization are separated, for example by decantation or filtration. The separated beads can be washed with water or an aqueous solution to remove traces of the water-immiscible liquid, and can be stored in an appropriate buffer.

Also part of the invention are polyacrylamide beads containing encapsulated cells obtainable by the process of the present invention. Preferably, the encapsulated cells are cells of a strain of the genus Rhodococcus containing a nitrile hydratase.

Another part of the invention is the use of above polyacrylamide beads containing encapsulated cells as a biocatalyst for the transformation of a substrate to a product Preferably, the substrate is a nitrile and the product is the corresponding amide. More preferably the substrate is 3-cyanopyridine and the product is nicotinamide.

Examples of nitriles are cyanamide, cyanoacetic acid, malonodinitrile, cyanoacetic acid methyl ester, acrylonitrile, butyronitrile, valeronitrile, crotononitrile, methacrylonitrile, 2-cyanopyridine, 3-cyanopyridine, 4-cyanopyridine, benzonitrile, 2-chlorobenzonitrile, 4-chlorobenzonitrile, pyrazinecarbonitrile, pyrazine-2,3dicarbonitrile, 2-furonitrile, thiophene-2-carbonitrile, pivalonitrile and cyclopropanecarbonitrile.

The transformation can be carried out as a batch reaction or as a continuous reaction. Preferably, the reaction is carried out in a suitable buffer at a temperature from 10 to 35° C.

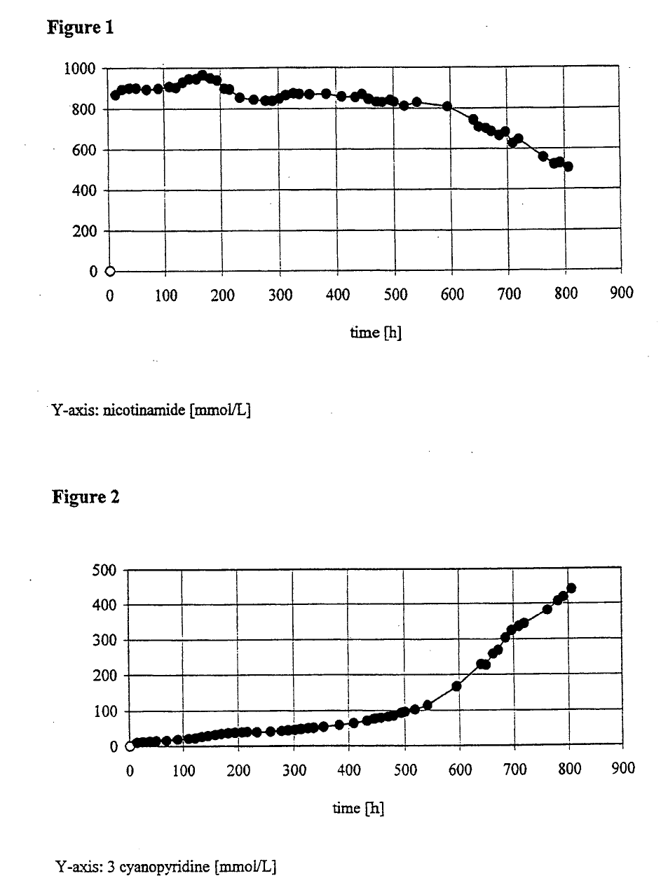

FIG. 1 shows the concentration of nicotinamide in the reaction mixture in dependency on the time during a continuous reaction of 3-cyanopyridine to nicotinamide.

FIG. 2 shows the concentration of 3-cyanopyridine in the reaction mixture in dependency on the time during a continuous reaction of 3-cyanopyridine to nicotinamide.

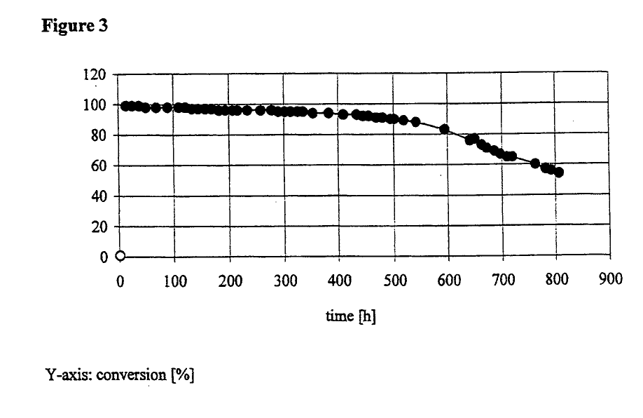

FIG. 3 shows the conversion of 3-cyanopyridine to nicotinamide in dependency on the time during a continuous reaction of 3-cyanopyridine to nicotinamide.

### EXAMPLE 1

### Cultivation of a Strain of the Genus Rhodococcus

1.1. Preparation of a Preculture

A sterile medium (200 mL, pH 7.0) containing 1.25% (w/w) yeast extract, 0.05% (w/w) MgSO₄.7 H₂O, 0.003% (w/w) CoCl₂.6 H₂O, 0.5% (w/w) sodium citrate, 0.75% (w/w) methacrylamide and 0.2% (w/w) KH₂PO₄ was inoculated with an agar plate culture of a stain of the genus Rhodococcus. The preculture was cultivated in an Erlenmeyer flask (500 mL) at 28° C. and 120 rpm for 48 h.

1.2. Preparation of a Culture

A sterile medium (12 L, pH 7.0) containing 1.25% (w/w) yeast extract, 0.05% (w/w) MgSO₄.7 H₂O, 0.003% (w/w) CoCl₂.6 H₂0, 0.5% (w/w) sodium citrate, 0.75% (w/w) methacrylamide and 0.2% (w/w) KHPO₄ was inoculated with a preculture (200 mL) of the strain of the genus Rhodococcus obtained as described in example 1.1. The culture was cultivated in a fermenter (12 L) at 28° C., pH 7.0, dissolved oxygen concentration >40% (in respect to the dissolved oxygen concentration at 1 volume air/(volume fermentation broth×min), 28° C.) and 300-400 rpm for 48 h. The cells were harvested by centrifugation, washed with phosphate buffer (50 nM, pH 7.0), concentrated to a concentration of dry cells of 15-20% (w/w) and stored at −40° C.

### EXAMPLE 2

### Nitrile Hydratase Activity Assay of a Strain of the Genus Rhodococcus

Polyacrylamide beads containing encapsulated cells of a strain of the genus Rhodococcus (0.2 g wet weight) were added to a solution of 3-cyanopyridine (1.59 g) in phosphate puffer (0.05 M, pH 7.0, 30 mL) at 25° C. Samples (1000 μl) were taken after 5 and 15 minutes. These samples were immediately mixed with 20 μl of H₂SO₄ (48 % (w/w)), diluted 100 times by volume with a mixture of methanol/water=40:60 (v/v), filtered (0.2 μm pore size) and analyzed by HPLC (column: C8 reverse phase, flow rate: 1 mL/min, mobile phase: methanol/water=40:60 (v/v)), wavelength: 210 nm, 25° C.). Dry polyacrylamide beads were obtained after drying the wet biocatalyst at 55° C. and 20 mbar for 4 h.

### EXAMPLE 3

### Encapsulation of Cells of a Strain of the Genus Rhodococcus in Polyacrylamide Beads

Acrylamide (42.25 g, 594 mmol), N,N′-methylenebisacrylamide (3.75 g, 24 mmol) and 2-(dimethylamino)ethyl methacrylate (1.5 g, 9 mmol) were dissolved in phosphate buffer (37.5 g, 50 mM, pH 7.0) and the pH of the solution was adjusted to 7.0. A solution of ammonium persulfate (0.465 g, 2 mmol) in distilled water (5 g) was added to a suspension of cells of a strain of the genus Rhodococcus (20% (w/w) dry cells, 165 g) obtained as described in example 1. A solution of N,N,N′,N′-tetranethylethylenediamine (0.232 g, 2 mmol) in distilled water (5 g) was dispersed in mineral oil (Exxsol D100, 350 g) in a reactor (1 L) at 350 rpm. The monomer solution, the cell suspension and the oil were separately purged with nitrogen for 15 min. The monomer solution (flow rate: 2.5 g/min) and the cell suspension (flow rate: 5 g/min) were separately pumped in a 2.5 mL mixing flask. The resulting mixture was immediately dropped in the stirred (350 rpm, visco-jet® stirrer) oil at 20° C. After complete addition the reaction mixture was stirred for further 3.5 h at 20° C. The obtained polyacrylamide beads containing encapsulated cells of a strain of the genus Rhodococcus were separated by filtration, washed with distilled water and allowed to swell in water. The polyacrylamide beads were stored in twice the amount by volume of a storage buffer (3.55 g/L sodium sulfate, 0.25% (w/w) dehydroacetic acid, sodium salt, 0.05% (w/w) nicotinamide, pH 7.0) at 4° C. The swollen beads were of regular spherical shape with a size of 200 μm to 1200 μm and a mechanical strength of >300 mN. The ratio of dry polyacrylamide beads/wet polyacrylamide beads was 0.11:1 (w/w). The specific activity was 9.5 μmol nicotinamide/(min×mg dry polyacrylamide beads).

### EXAMPLE 4

### Conversion of 3-cyanopyridine to Nicotinamide, Batch Reaction

Polyacrylamide beads containing encapsulated cells of a strain of the genus Rhodococcus (100 g wet weight) obtained as described in example 3 were added to a gently stirred solution of 3-cyanopyridine (40 g, 3.8 mol) in phosphate buffer (0.05 M, pH 7.0, 400 mL) at 25° C. After 15 min 99% of 3-cyanopyridine was converted to nicotinamide, after 30 min 99% of 3-cyanopyridine was converted to nicotinamide.

### EXAMPLE 5

### Conversion of 3-cyanopyridine to nicotinamide, Continuous Reaction

Polyacrylamide beads containing cells of a strain of the genus Rhodococcus (100 g wet weight) obtained as described in example 3 were added to a solution of 3-cyanopyridine (40 g, 3.8 mol) in phosphate buffer.(0.05 M, pH 7.0, 400 mL) at 25° C. A solution of 3-cyanpyridine (10% (w/w)) in phosphate buffer (0.05 M, pH 7.0) was continuously added to the gently stirred reaction mixture, and reaction mixture (without polyacrylamide beads) was continuously removed. The continuous conversion was performed with a retention time-of 3.1 h for 5 weeks at 25° C. No abrasion of the beads was observed after 5 weeks. The concentrations of 3-cyanopyridine and nicotinamide were determined (see FIGS. 1 and 2) and the conversion calculated (see FIG. 3).

### EXAMPLE 6

### Encapsulation of Cells of a Strain of the Genus Rhodococcus in Polyacryamide Beads

Acrylamide (422.5 g, 5940 mmol), N,N′-methylenebisacrylamide (37.5 g, 240 mmol) and 2-dimethylamino)ethyl methacrylate (15 g, 90 mmol) were dissolved in phosphate buffer (375 g, d50 mM, pH 7.0) and the pH of the solution was adjusted to 7.0. A solution of ammonium persulfate (4.65 g, 20 mmol) in distilled water (25 g) was added to a suspension of cells of a strain of the genus Rhodococcus (16% (w/w) dry cells, 1650 g) obtained as described in example 1. A solution of N,N,N′,N′-tetramethylethyienediamine (2.32 g, 20 mmol) in distilled water (25 g) was dispersed in mineral oil (Exxsol D100, 3500 g) in a reactor (10 L). The monomer solution, the cell suspension and the oil were separately purged with nitrogen for 15 min. The monomer solution (flow rate: 13.5 g/min) and the cell suspension (flow rate: 27 g/min) were separately pumped in a common tubing. The resulting mixture was pumped in the stirred (215 rpm, visco-jet® stirrer) oil at 20° C. After complete addition the reaction mixture was stirred for further 3.5 h at 20° C. The obtained polyacrylamide beads containing encapsulated cells of a strain of the genus Rhodococcus were separated, washed and stored as described in example 3. The swollen beads were of regular spherical shape, with a size of 200 μm to 1200 μm and a mechanical strength of >400 mN. The ratio dry polyacrylamide beads/wet polyacrylamide beads was 0.09:1 (w/w). The specific activity was 7.3 μmol nicotinamide/(min×mg dry polyacrylamide beads).

### EXAMPLE 7

### Storage Stability of Polyacrylamide Beads Containing Encapsulated Cells of a Stain of the Genus Rhodococcus

Polyacrylamide beads containing encapsulated cells of a strain of the genus Rhodococcus obtained as described in example 5 were stored in an aqueous storage solution (3.55 g/L sodium sulfate, 0.25% (w/w) sodium dehydroacetic acid, sodium salt, 0.05% (w/w) nicotinamide, pH 7.0) at 4° C. for 50 weeks. Samples were taken every fifth week. The polyacrylamide beads were separated, washed with distilled water, and suspended in fresh storage solution (3.55 g/L sodium sulfate, 0.25% (w/w) dehydroacetic acid, sodium salt, 0.05% (w/w) nicotinamide, pH 7.0) at 25° C. for 1 h. The nitrile hydratase activity was determined as described in example 2. The ratio of dry polyacrylamide beads/wet polyacrylamide beads were determined. Dry polyacrylamide beads were obtained after drying the wet polyacrylamide beads at 55° C. and 20 mbar for 4 h.

TABLE 1
storage stability of polyacrylamide beads containing cells of the genus
Rhodococcus

| | dry polyacrylamide beads/ | Specific activity |
|---|---|---|
| | wet polyacrylamide beads | [μmol nicotinamide/(min × mg |
| week | (w/w) | dry polyacrylamide beads)] |
| 0 | 0.09 | 7.3 |
| 5 | 0.09 | 7.3 |
| 10 | 0.09 | 7.0 |
| 13 | 0.09 | 6.5 |
| 50 | 0.08 | 6.0 |

### EXAMPLE 8

### Encapsulation of Cells of a Strain of the Genus Rhodococcus in Polyacrylamide Beads

The incapsulation was performed in analogy to the encapsulation described in example 3, except that a solution of ammonium persulfate (1.86 g, 8 mmol) in distilled water (7.0 g) and a solution of N,N,N′,N′-tetramethylethylenediamine (0.928 g, 8 mmol) in distilled water (5 g) were employed. The obtained polyacrylamide beads containing encapsulated cells of a strain of the genus Rhodococcus were separated, washed and stored as described in example 3. The swollen beads were of regular spherical shape, with a size of 250 μm to 1300 μm and a mechanical strength of >400 mN. The swelling ratio of dry poly-acrylamide beads/wet polyacrylamide beads was 0.12:1 (w/w). The specific activity was 7.8 μmol nicotinamide/(min×mg polyacrylamide beads).

### EXAMPLE 9

### Encapsulation of Cells of a Strain of the Genus Rhodococcus in Polyacrylamide Beads

The encapsulation was performed in analogy to the encapsulation described in example 3, except that a suspension of cells of a strain of the genus Rhodococcus (16% (w/w) dry cells) employed, and the polymerization was performed at 10° C. for 9 h. The obtained polyacrylamide beads containing encapsulated cells of a strain of the genus Rhodococcus were separated, washed and stored as described in example 3. The swollen beads were of regular spherical shape, with a diameter from 250 μm to 1300 μm and a mechanical strength of >400 mN. The ratio of dry polyacrylamide beads/wet poly-acrylamide beads was 0.09:1.00 (w/w). The specific activity was 7.3 μmol nicotin-amide/(min×mg dry polyacrylamide beads).

### EXAMPLE 10

### Encapsulation of Cells of a Strain of the Genus Rhodococcus in Polyacrylamide Beads

N,N-Dimethylacrylamide (42.25 g, 426 mmol), N,N′-methylenebisacrylamide (3.75 g, 24 mmol) and 2-dimethylamino)ethyl methacrylate (1.5 g, 9 mmol) were dissolved in phosphate buffer (37.5 g, 50 mM, pH 7.0) and the pH of the solution was adjusted to 7.0. A solution of ammonium persulfate (1.86 g, 8 mmol) in distilled water (7 g) was added to a suspension of cells of a strain of the genus, Rhodococcus (18% (w/w) dry cells, 165 g) prepared as described in example 1. A solution of N,N,N′,N′-tetramethylethylenediamine (0.928 g, 8 mmol) in distilled water (7 g) was dispersed in mineral oil (Exxsol D100, 350 g) in a reactor (1 L). The monomer solution, the cell suspension and the oil were separately purged with nitrogen for 15 min. The monomer solution (flow rate: 2.5 g/min) and the cell suspension (flow rate: 5 g/min) were separately pumped in a 2.5 mL mixing flask. The resulting mixture was immediately dropped in the stirred (350 rpm, visco-jet® stirrer) oil at 20° C. After complete addition the reaction mixture was stirred for further 3.5 h at 20° C. The obtained polyacrylamide beads containing encapsulated cells of a strain of the genus Rhodococcus were separated by filtration, washed and stored as described in example 3. The swollen beads were of regular spherical shape with a size of 200 μm to 700 μm and a mechanical strength of >400 mN. The ratio of dry polyacrylamide beads/wet polyacrylamide beads was 0.21:1 (w/w). The specific activity was 5.4 μmol nicotinamide/(min×mg dry, polyacrylamide beads).

### EXAMPLE 11

### Encapsulation of Cells of a Strain of the Genus Rhodococcus in Polyacrylamide Beads

The encapsulation was performed in analogy to the encapsulation described in example 10 except that acrylamide (42.25 g, 594 mmol) instead of N,N-dimethylacrylamide (42.25 g, 426 mmol) and N-[3-(dimethylamino)propyl]methacrylamide (1.5 g, 9 mmol) instead of 2-(dimethylamino)ethyl methacrylate (1.5 g, 9 mmol) were employed. The obtained polyacrylamide beads containing encapsulated cells of a strain of the genus Rhodococcus were separated, washed and stored as described in example 3. The swollen beads were of regular spherical shape with a size of 150 μm to 1200 μm and a mechanical strength of >400 mN. The ratio of dry polyacrylamide beads/wet polyacrylamide beads was 0.13:1 (w/w). The specific activity was 5.9 μmol nicotinamide/(min×mg dry polyacrylamide beads).

### EXAMPLE 12

### Cultivation of a Strain of the Species Escherichia coli Containing a Plasmid Having a Gene Encoding for an Amidase Under the Transcriptional Control of the Rhamnose Promoter

12.1. Preparation of a Pre-Preculture

A sterile medium (5 mL, pH 7.0) containing 1.6% (w/w) tryptone, 1.0% (w/w) yeast extract, 0.5% (w/w) NaCl and 0.01% (w/w) ampicillin was inoculated with a agar plate culture of a strain of the species Escherichia coli containig a plasmid having a gene encoding for an amidase under the transcriptional control of the rhamnose promoter. The pre-preculture was cultivated at 37° C. for 12 h on a shaker.

12.2. Preparation of a Preculture

The sterile medium described in example 12.1 (100 mL) was inoculated with 5 mL of a pre-preculture of the strain of the species Escherichia coli obtained as described in example 12.1. The preculture was cultivated at 37° C. on a shaker. At OD₆₀₀ 0.25, 0.2% (w/w) L-rhamnose was added to the culture. At OD₆₀₀ 5, the cells were harvested by centrifugation, washed twice with buffer (1.80 g/L ethylenediaminetetraacetic acid, 2.65 g/L disodium salt/sodium acetate buffer, pH 7.0) and resuspended in the same buffer to a dry cell concentration of 15-20% (w/w). The cell suspension was stored at −40° C.

### EXAMPLE 13

### Amidase Assay

Polyacrylamide beads containing encapsulated cells of a strain of the genus Escherichia containing an amidase (0.4 g wet weight) were added to a stirred solution of 2-hydroxy-2-methyl-3,3,3-trifluoropropionamide (1.0 g) in phosphate puffer (0.1 M, pH 8.0, 9 mL) at 37° C. Samples (200 μl) were taken after 0, 30 and 60 minutes. The molar amount of formed ammonia was measured. The molar amount of formed ammonia equals the molar amount of formed 2-hydroxy-2-methyl-3,3,3-trifluoropropionic acid.

### EXAMPLE 14

### Encapsulation of Strain of the Species Escherichia Coli Containing a Plasmid Having a Gene Encoding for an Amidase Under the Transcriptional Control of the Rhamnose Promoter in Polyacrylamide Beads

The encapsulation was performed in analogy to the encapsulation described in example 3, except that a suspension of cells of a strain of the species Escherichia coli (19% (w/w) dry cells) obtained as described in example 12, a solution of ammonium persulfate (1.86 g, 8 mmol) in distilled water (7.0 g) and a solution of N,N,N′,N′-tetramethylethylenediamine (0.928 g, 8 mmol) in distilled water (5 g) were employed, and the polymerization was performed at 400 rpm (visco-jet® stirrer). The obtained polyacrylamide beads containing encapsulated cells of a strain of the species Escherichia coli were separated and washed as described in example 3 and stored in phosphate buffer (0.1 M, pH 7.0) at 4° C. The swollen beads were of irregular spherical shape, with a size of 200 μm to 2000 μm and a mechanical strength of >200 mN. The ratio of dry polyacrylamide beads/wet polyacrylamide beads was 0.2 1:1 (w/w). The specific activity was 0.029 μm 2-hydroxy-2-methyl-3,3,3-trifluoropropionamide/(min×mg dry polyacrylamide beads).

### EXAMPLE 15

### Conversion of 2-hydroxy-2-methyl-3,3,3-trifluoropropionamide to 2-hydroxy-2-methyl-3,3,3-trifluoropropionic acid, Batch Reaction

Polyacrylamide beads containing cells of a strain of the species Escherichia coli containing a plasmid having a gene encoding for an amidase obtained as described in example 14 (0.4 g wet weight) were added to a solution of 2-hydroxy-2-methyl-3,3,3-tri-fluoropropionamide (1.0 g, 6.366 mmol) in phosphate buffer (0.1 M, pH 8.0, 10 mL) at 37° C. for 1 h. 2-Hydroxy-2-methyl-3,3,3-trifluoropropionic acid (2%) was formed.

### EXAMPLE 16

### Encapsulation of a Strain of the Species Escherichia Coli Containing a Plasmid Having a Gene Encoding for an Amidase Under the Transcriptional Control of the Rhamnose Promoter in Polyacrylamide Beads

Acrylamide (21.13 g, 297 mmol), N,N-methylenebisacrylamide (1.88 g, 12 mmol) and 2-(dimethylamino)ethyl methacrylate (0.75 g, 4.8 mmol) were dissolved in phosphate buffer (18.75 g, 50 mM, pH 7.0) and the pH of the solution was adjusted to 7.0. A solution of ammonium persulfate (0.93 g, 4 mmol) in distilled water (2.5 g) was added to a suspension of cells of a strain of the species Escherichia coli (19% (w/w) dry cells, 82.5 g) obtained as described in example 12. A solution of N,N,N′,N′-tetramethylethylenediamine (0.928 g, 8 mmol) in distilled water (5 g) was dispersed in mineral oil (Isopar M, 350 g) in a reactor (1 L) at 450 rpm. The monomer solution, the cell suspension and the oil phase were separately purged with nitrogen for 15 min. The monomer solution (flow rate: 2.5 g/min) and the cell suspension (flow rate: 5 g/min) were separately pumped in a 2.5 mL mixing flask. The resulting mixture was immediately dropped in the stirred (450 rpm, visco-jet® stirrer) oil at 20° C. After complete addition the reaction mixture was stirred for further 3.75 h at 20° C. The obtained polyacrylamide beads containing encapsulated cells of a strain of the species Escherichia coli were separated and washed as described in example 3, and stored in phosphate buffer (0.1 M, pH 7.0) at 4° C. The swollen beads were of irregular spherical shape with size of 1000 μm to 2000 μm and a mechanical strength of >200 mN. The ratio of dry polyacrylamide beads/wet polyacrylamide beads was 0.25:1.00 (w/w). The specific activity was 0.016 μmol nicotinamide(min×mg dry polyacrylamide beads).

### EXAMPLE 17

### Use of the Polyacrylamide Beads Containing Encapsulated Cells of the Genus Rhodococcus Containing a Nitrile Hydratase as a Biocatalyst for the Conversion of Nitriles to Amides

Polyacrylamide beads containing encapsulated cells of the genus Rhodococcus obtained as described in example 7 (25 g wet weight) were added to a gently stirred solution of a nitrile in phosphate buffer (0.05 M, pH 7, 100 mL) or in a mixture of phosphate buffer (0.05 M, pH 7, 100 mL) and methanol at 25° C. Samples (3 mL) were taken after 5, 15 and 60 minutes and mixed immediately with H₂SO₄ (48% (w/w), 0.03 mL). The reaction mixture was analyzed by HPLC or GC. The specific activity was determined. The results are given in Table 2.

TABLE 2
Biotransformation of various nitriles to the corresponding amides using
polyacrylamide beads containing cells of the genus Rhodococcus
containing a nitrile hydratase as the biocatalyst.

| | | | ratio | specific |
|---|---|---|---|---|
| | | concentration | MeOH/ | activity |
| | | substrate | buffer | [μmol/ |
| substrate | product | [mM] | [v/v] | (min × mg)] |
| cyanamide | urea | 200 | 0:1 | 857 |
| cyanoacetic acid | malonamic acid | 100 | 0:1 | 107 |
| malonodinitrile | 2-cyanoacetamide/ | 200 | 0:1 | 946 |
| | malonamide | | | |
| cyanoacetic acid | malonic acid methyl | 100 | 0:1 | 340 |
| methyl ester | ester | | | |
| acrylonitrile | acrylamide | 200 | 0:1 | 76 |
| butyronitrile | butyramide | 200 | 0:1 | 1025 |
| valeronitrile | valeramide | 200 | 1:9 | 1708 |
| crotononitrile | crotonamide | 200 | 0:1 | 1585 |
| methacrylonitrile | methacrylamide | 200 | 0:1 | 591 |
| 2-cyanopyridine | picolinamide | 9.6 | 0:1 | 24.6 |
| 3-cyanopyridine | nicotinamide | 250 | 0:1 | 2320 |
| 4-cyanopyridine | isonicotinamide | 125 | 0:1 | 613 |
| benzonitrile | benzamide | 50 | 1:4 | 276 |
| 2-chlorobenzonitrile | 2-chlorobenzamide | 7.3 | 1:4 | 6.4 |
| 4-chlorobenzonitrile | 4-chlorobenzamide | 7.2 | 1:4 | 42.6 |
| pyrazinecarbonitrile | pyrazine-2-carboxamide | 100 | 1:4 | 246 |
| pyrazine-2,3- | pyrazine-2,3- | 7.7 | 1:4 | 0.53 |
| dicarbonitrile | dicarboxamide | | | |
| 2-furonitrile | furan-2-carboxamide | 100 | 1:4 | 235 |
| thiophene-2- | thiophene-2- | 9.2 | 1:4 | 73 |
| carbonitrile | carboxamide | | | |
| pivalonitrile | 2,2-dimethyl- | 100 | 1:4 | 321 |
| | propionamide | | | |
| cyclopropanecarbonitrile | cyclopropane- | 100 | 1:4 | 562 |
| | carboxamide | | | |

1. A process for the preparation of polyacrylamide beads containing encapsulated cells comprising the steps of
(i) providing an aqueous solution of a mixture of acrylic monomers,
(ii) providing a suspension of cells in an aqueous solution of a persulfate,
(iii) providing an emulsion of an aqueous solution of a tertiary amine in an water-immiscible liquid, which liquid optionally contains a surfactant,
(iv) mixing the solution provided in step (i) and the suspension provided in step (ii),
(v) adding the mixture obtained in step (iv) to the stirred emulsion provided in step (iii),
(vi) polymerizing the mixture of acrylic monomers and simultaneously encapsulating the cells to form polyacrylamide beads containing encapsulated cells.

2. The process of claim 1 wherein the polyacrylamide beads have a size of 0.05 to 3 mm and a mechanical strength of at least 200 mN.

3. The process of claim 2 wherein the polyacrylamide beads have a size of 0.1 to 1.5 mm and a mechanical strength of at least 300 mN.

4. The process of claim 1, wherein the ratio of dry cells/mixture of acrylic monomers is 0.001:1 to 1:1 (w/w).

5. The process of claim 1, wherein the ratio of dry cells/mixture of acrylic monomers is 0.2:1 to 0.9:1 (w/w).

6. The process of claim 1 wherein the cell is a bacterial cell.

7. The process of claim 6 wherein the cell is a cell of a bacterium of the group nocardioform Actinomycetes or of the family Enterobacteriaceae.

8. The process of claim 1 wherein the tertiary amine is N,N,N′,N′-tetramethylethylenediamine or 3-(dimethylamino)propionitrile.

9. The process of claim 1 wherein the water-immiscible liquid is a mineral oil.

10. The process of claim 1 wherein no surfactant is used.

11. The process of claim 1 wherein the polyacrylamide beads formed in step (vi) are separated.

12. Polyacrylamide beads containing encapsulated cells obtainable by a process of claim 1.

13. The polyacrylamide beads of claim 12 wherein the encapsulated cells are cells of a strain of the genus Rhodococcus containing a nitrile hydratase.

14. Method of biocatalysis, comprising the step of contacting the polyacrylamide beads of claim 12 with a substrate to biocatalytically transform the substrate to a product.

15. Method of claim 14, wherein a nitrile is transformed to the corresponding amide.

16. Method of claim 15, wherein 3-cyanopyridine is transformed to nicotinamide.
